(19) United States
(12) Patent Application Publication
(10) Pub. No.: US 2026/0183327 A1
(43) Pub. Date: Jul. 2, 2026

(54) COMPOSITION FOR PREVENTION OR TREATMENT OF AUTOIMMUNE NEUROLOGICAL DISORDER, COMPRISING PLGA-SISTAT3 AS ACTIVE INGREDIENT

(71) Applicant: UNIVERSITY-INDUSTRY COOPERATION GROUP OF KYUNG HEE UNIVERSITY, Yongin-si, Gyeonggi-do (KR)

(72) Inventors: Ik-Hyun CHO, Seoul (KR); Tae Woo KWON, Seoul (KR)

(73) Assignee: UNIVERSITY-INDUSTRY COOPERATION GROUP OF KYUNG HEE UNIVERSITY, Yongin-si, Gyeonggi-do (KR)

(21) Appl. No.: 19/128,657

(22) PCT Filed: Nov. 10, 2023
(86) PCT No.: PCT/KR2023/018055

§ 371 Date: May 9, 2025

(30) Foreign Application Priority Data
Nov. 10, 2022 (KR) 10-2022-0149680
Nov. 10, 2023 (KR) 10-2023-0155187

(51) Int. Cl. A61K 31/7105; A61K 9/51; A61P 25/00

(52) U.S. Cl. CPC A61K 31/7105; A61K 9/5153; A61P 25/00

(57) ABSTRACT

The present invention relates to a composition for prevention or treatment of autoimmune neurological disorder, comprising nanoparticles (PLGA-siSTAT3) comprising STAT3-specific siRNA and a biodegradable polymer material, the present invention having confirmed that, in an EAE animal model, which is a multiple sclerosis or encephalomyelitis animal model, the PLGA-siSTAT3 reduces a clinical symptom index induced by EAE, and inhibits demyelination of the spinal cord. In addition, the present invention has been confirmed to block a STAT3 signaling pathway associated with EAE and thus reduce demyelination, and inhibit microglial and neuroglial expression. In addition, the present invention has been confirmed to inhibit the inflammatory factors iNOS, IL-1β, IL-6, TNF-α, and inhibit the expression of the inflammation-mediating enzyme COX-2 and the chemokines MIP-1α, RANTES. In addition, the present invention has been confirmed to inhibit the invasion and activity of Th1 and Th17 cells associated with autoimmunity and thus enable reducing autoimmune neurological disorder.

### CROSS-REFERENCE TO RELATED APPLICATIONS

This is a National Stage of International Application No. PCT/KR2023/018055 filed Nov. 10, 2023, claiming priority based on Korean Patent Application No. 10-2022-0149680 filed Nov. 10, 2022 and Korean Patent Application No. 10-2023-0155187 filed Nov. 10, 2023.

### INCORPORATION BY REFERENCE OF SEQUENCE LISTING

The instant application contains a Sequence Listing which has been filed electronically in xml format and is hereby incorporated by reference in its entirety. Said xml file, created on May 5, 2025, is named Q307958_sequence listing as filed .xml and is 20,115 bytes.

### TECHNICAL FIELD

The present disclosure relates to a composition for prevention or treatment of autoimmune neurological disorder, comprising nanoparticles PLGA-siSTAT3 consisting of STAT3-specific siRNA and a biodegradable polymer material as an active ingredient.

### BACKGROUND ART

Immune diseases are diseases in which components of the immune system cause, mediate, or otherwise contribute to the pathological conditions, and particularly, inflammatory disorder is one of the most important health problems worldwide. Inflammation generally refers to a localized protective response of body tissues to host invasion by foreign materials or harmful stimuli. Causes of inflammation include infectious causes, such as bacteria, viruses, and parasites; physical causes, such as burns or radiation; chemical agents, such as toxins, drugs, or industrial agents; immune responses, such as allergies and autoimmune reactions, or conditions associated with oxidative stress.

In addition, autoimmune disease, one type of immune disease, is characterized by the immune system attacking its own organs and causing a spontaneous response. These responses are caused by recognition of auto-antigens by T lymphocytes, which induces humoral (auto-antigen production) and cellular (increased cytotoxic activity of lymphocytes and macrophages) immune responses. The autoimmune disease includes rheumatoid disease, psoriasis, systemic dermatomyositis, multiple sclerosis, lupus erythematosus, or deterioration of immune responses by antigens, such as asthma, allergies to drugs or foods, etc. These diseases are all limited and chronic diseases, and in some cases, fatal, and currently, there is no effective treatment method to treat the diseases. Therefore, any drug, medicine, or medium capable of reducing or alleviating the disease during the progression of the corresponding disease may be considered an important solution for the patient's health.

Meanwhile, multiple sclerosis is a disease caused by a defect of the immune system in the human body, and sporadically causing demyelination by invading the myelin sheath consisting of fat that covers the nerves in the white matter of the brain and spinal cord. When the myelin sheath is damaged and the thickness of the myelin layer decreases, nerve impulses are transmitted, but the efficiency decreases, and normal nerve impulse transmission is disrupted to cause the disease. Simply, just as the conduction of electricity slows down when an insulator surrounding an electric wire is damaged, the conduction speed between nerve cells slows down to cause symptoms. Initially, symptoms may be alleviated as the myelin sheath recovers, but eventually, the symptoms were worsened repeatedly and the damage becomes permanent. The nerves are not properly functioned due to damage of these myelin sheaths to cause symptoms such as visual disturbances, loss of balance, and the like, and cause paralysis in some patients.

Encephalomyelitis is a demyelinating disease. The myelin sheath is a sheath-like part that wraps around the axon centralized in the nerve fiber, and the selective loss of only the myelin sheath is called demyelination. Primary white matter (part of a group of nerve fibers in the central nervous system of higher animals) disease characterized by demyelination and accompanying some inflammation is called demyelinating disease. Encephalomyelitis causes inflammation in both the brain and spinal cord, and is caused by microbial or viral infection, and refers to a condition with inflammatory changes characterized by degenerative changes in neural tissue components, cellular and fluid exudation from the blood, pericyte invasion, and proliferation of neuroglia and vascular connective tissue. Acute disseminated encephalomyelitis is also referred to as acute demyelinating encephalomyelitis, and is a dehydrating disease in which acute inflammation is scattered throughout the central nervous system centered on the spinal cord and a disease occurring after vaccination or suffering from infectious diseases. The main causes are vaccinations and infections related to various viruses or bacteria that may easily cause disorders in the nervous system, and may occur after vaccinations for rabies, smallpox, influenza, polio, tetanus, measles, and whooping cough, and may also occur after suffering from measles, mumps, chickenpox, and smallpox, in addition to vaccination. The headache, stiff neck, or the like starts within 2 to 4 days of having viral infection or 10 to 13 days after vaccination, followed by paralysis and coma, and has a mortality rate of 30 to 50%.

Treatment for multiple sclerosis or encephalomyelitis may be broadly divided into acute treatment, long-term disease-relieving treatment, and symptom-relieving treatment. In an acute phase, intravenous therapy of high-dose steroid is usually used. The steroid relieves symptoms in the acute phase and shortens a recovery period, but needs to be used with caution because the steroid may cause various side effects when administered for a long time. Patients who have difficulty in receiving palliative treatment for various causes may also use low-dose steroid maintenance therapy. Representative disease-alleviating therapeutic agents include beta-interferon and glatiramer acetate. These therapeutic agents have effects of reducing the number of recurrences in relapsing-remitting multiple sclerosis (RRMS) and alleviating symptoms that occur during recurrence. However, since no significant effect is observed in patients who have transitioned to the secondary progressive type, in this case, early treatment is most important to prevent disorders of the patient. Symptom-alleviating treatment is also performed to reduce various symptoms and neurological disorders caused by multiple sclerosis or encephalomyelitis. These treatments have a high recurrence rate, and there is no method for definitely preventing recurrence.

Meanwhile, poly(lactic-co-glycolic acid) (PLGA) is a biodegradable and biocompatible polymer material that is degraded into lactic acid and glycolic acid in the body and ultimately released into carbon dioxide and water, and is approved by the U.S. Food and Drug Administration. In addition, a technology for preparing biodegradable polymer nano-/microparticles has recently attracted much attention through research on vaccine manufacture, bioimmunological disease treatment, etc., as well as use as delivery systems.

### DISCLOSURE

### Technical Problem

An object of the present disclosure is to provide a pharmaceutical composition for prevention or treatment of autoimmune neurological disorder.

Another object of the present disclosure is to provide a food composition for prevention or alleviation of autoimmune neurological disorder.

Yet another object of the present disclosure is to provide a health functional food composition for prevention or alleviation of autoimmune neurological disorder.

Still another object of the present disclosure is to provide a method for preventing or treating autoimmune neurological disorder.

### Technical Solution

To achieve the object, an aspect of the present disclosure provides a pharmaceutical composition for prevention or treatment of autoimmune neurological disorder, including nanoparticles consisting of STAT3-specific siRNA and a biodegradable polymer material.

To achieve another object, another aspect of the present disclosure provides a food composition for prevention or alleviation of autoimmune neurological disorder, including nanoparticles consisting of STAT3-specific siRNA and a biodegradable polymer material.

To achieve yet another object, yet another aspect of the present disclosure provides a healthy functional food composition for prevention or alleviation of autoimmune neurological disorder, including nanoparticles consisting of STAT3-specific siRNA and a biodegradable polymer material.

To achieve still another object, still another aspect of the present disclosure provides a method for preventing or treating autoimmune neurological disorder, including administering to a subject nanoparticles consisting of STAT3-specific siRNA and a biodegradable polymer material.

### Advantageous Effects

The present disclosure relates to a composition for prevention or treatment of autoimmune neurological disorder, including nanoparticles (PLGA-siSTAT3) consisting of STAT3-specific siRNA and a biodegradable polymer material. According to the present disclosure, it was confirmed that in an EAE animal model, which is a multiple sclerosis or encephalomyelitis animal model, PLGA-siSTAT3 reduced a clinical symptom index induced by EAE, and inhibited demyelination of the spinal cord. In addition, it was confirmed that PLGA-siSTAT3 blocked a STAT3 signaling pathway associated with EAE and thus reduced demyelination and inhibit microglial and neuroglial expression. In addition, it was confirmed that PLGA-siSTAT3 inhibited the inflammatory factors iNOS, IL-1β, IL-6, and TNF-α, and inhibited the expression of the inflammation-mediating enzyme COX-2 and the chemokines MIP-1α and RANTES. In addition, it has been confirmed that PLGA-siSTAT3 inhibits the invasion and activity of Th1 and Th17 cells associated with autoimmunity to reduce autoimmune neurological disorder, and thus can be usefully used in related fields.

### DESCRIPTION OF DRAWINGS

FIG. 1 shows results of confirming effects of nanoparticles (P-siSTAT3) consisting of STAT3-specific siRNA and a biodegradable polymer material of the present disclosure treated to microglia inducing inflammatory responses for each concentration on the expression of inflammation-mediating enzymes, inflammatory cytokines, and major signaling mechanism factors. A of FIG. 1 shows a result of confirming the protein expression of each factor, B of FIG. 1 shows a result of confirming the expression of Iba-1, C of FIG. 1 shows a result of confirming the expression of iNOS, D of FIG. 1 shows a result of confirming the expression of IL-6, E of FIG. 1 shows a result of confirming the expression of TNF-α, F of FIG. 1 shows a result of confirming the expression of p-STAT3, and G of FIG. 1 shows a result of confirming the expression of STAT3. In FIG. 1, * means P>0.05, ** means p>0.01, ## means p>0.01, and ### means p>0.001.

FIG. 2 shows results of confirming an effect on the expression of blood-brain barrier (BBB)-related factors by treating P-siSTAT3 of the present disclosure in microglia with induced an inflammatory response for each concentration. A of FIG. 2 shows a result of confirming the protein expression of each factor, B of FIG. 2 shows a result of confirming the expression of PECAM-1, C of FIG. 2 shows a result of confirming the expression of claudin-5, and D of FIG. 2 shows a result of confirming the expression of ZO-1. In FIG. 2, * means P>0.05, ** means p>0.01, # means P>0.05, and ## means P>0.01.

FIG. 3 shows results of confirming an effect of improving clinical symptoms by administering P-siSTAT3 of the present disclosure for each concentration to an animal model of autoimmune neurological disorder. A of FIG. 3 shows a result of confirming an improvement effect of P-siSTAT3 for each concentration, and B of FIG. 3 shows comparing results of P-siSTAT3 and a control group. In FIG. 3, ** means P>0.01, and ### means P>0.001.

FIG. 4 shows results of confirming a demyelination inhibitory effect using luxol fast blue (LFB) staining by administering P-siSTAT3 of the present disclosure for each concentration to an animal model of autoimmune neurological disorder. A of FIG. 4 shows a result of confirming an inhibitory effect of P-siSTAT3 for each concentration, and B of FIG. 4 shows comparing results of P-siSTAT3 and a control group.

FIG. 5 shows results of confirming MBP, a myelin-forming protein, through immunofluorescence analysis by administering P-siSTAT3 of the present disclosure for each concentration to an animal model of autoimmune neurological disease. A of FIG. 5 shows a result of confirming an increasing effect of P-siSTAT3 for each concentration, and B of FIG. 5 shows comparing results of P-siSTAT3 and a control group.

FIG. 6 shows results of confirming the inhibition of STAT3 expression by immunofluorescence analysis by administering P-siSTAT3 of the present disclosure for each concentration to an animal model of autoimmune neurological disease. A of FIG. 6 shows a result of confirming an inhibitory effect of P-siSTAT3 for each concentration, and B of FIG. 6 shows comparing results of P-siSTAT3 and a control group.

FIG. 7 shows results of confirming an effect of inhibiting BBB damage by administering P-siSTAT3 of the present disclosure for each concentration to an animal model of autoimmune neurological disease. A of FIG. 7 shows a result of confirming the expression of GFAP, an activation marker of astrocytes, and PECAM-1, a cell adhesion protein, B of FIG. 7 shows a result of confirming the expression intensity of GFAP, C of FIG. 7 shows a result of confirming the expression intensity of PECAM-1, D of FIG. 7 shows a result of confirming the expression level of GFAP, E of FIG. 7 shows a result of confirming the expression level of PECAM-1, F of FIG. 7 shows a result of confirming the expression of cell junction proteins claudin-5, occludin, and ZO-1 (zonula occludens), G of FIG. 7 shows a result of confirming the expression intensity of claudin-5, H of FIG. 7 shows a result of confirming the expression intensity of occludin, I of FIG. 7 shows a result of confirming the expression intensity of ZO-1, J of FIG. 7 shows a result of confirming the expression of albumin, and K of FIG. 7 shows a result of confirming the expression intensity of albumin. In FIG. 7, ** means P>0.01, *** means P>0.001, ## means P>0.01, and ### means P>0.001.

FIG. 8 shows results of confirming the selectivity of P-siSTAT3 of the present disclosure for microglia. A of FIG. 8 shows a result of confirming the expression of coumarin-6 included in P-siSTAT3 in cells expressing Iba-1, a microglial marker, GFAP, an astrocyte marker, and Oligo-2, an oligodendrocyte marker, B of FIG. 8 shows comparing results for Iba-1 expressing cells in a control group and an EVE animal model, C of FIG. 8 shows comparing results for GFAP expressing cells in the control group and the EVE animal model, and D of FIG. 8 shows comparing results for Oligo-2 expressing cells in the control group and the EVE animal model. In FIG. 8, ### means P>0.001.

FIG. 9 shows results of confirming an effect of inhibiting immune cell invasion by H&E staining by administering P-siSTAT3 of the present disclosure for each concentration to an animal model of autoimmune neurological disease. A of FIG. 9 shows a result of confirming an inhibitory effect of P-siSTAT3 for each concentration, and B of FIG. 9 shows comparing results of P-siSTAT3 and a control group.

FIG. 10 shows results of confirming the expression of Iba-1, a microglial marker by immunofluorescence analysis by administering P-siSTAT3 of the present disclosure for each concentration to an animal model of autoimmune neurological disease. A of FIG. 10 shows a result of confirming an inhibitory effect of P-siSTAT3 for each concentration, and B of FIG. 10 shows comparing results of P-siSTAT3 and a control group.

FIG. 11 shows results of confirming an effect of inhibiting the expression of inflammatory proteins by Western blot by administering P-siSTAT3 of the present disclosure for each concentration to an animal model of autoimmune neurological disease. A of FIG. 11 shows a result of confirming an inhibitory effect of P-siSTAT3 for each concentration on the expression of each factor, B of FIG. 11 shows comparing results of P-siSTAT3 and a control group, C of FIG. 11 shows a result of confirming the expression of STAT3 in a P-siSTAT3 group, D of FIG. 11 shows a result of confirming the expression of pSTAT3 in the P-siSTAT3 group, E of FIG. 11 shows a result of confirming the expression of GFAP in the P-siSTAT3 group, F of FIG. 11 shows a result of confirming the expression of Iba-1 in the P-siSTAT3 group, G of FIG. 11 shows a result of confirming the expression of iNOS in the P-siSTAT3 group, H of FIG. 11 shows a result of confirming the expression of STAT3 in the control group, I of FIG. 11 shows a result of confirming the expression of pSTAT3 in the control group, J of FIG. 11 shows a result of confirming the expression of GFAP in the control group, K of FIG. 11 shows a result of confirming the expression of Iba-1 in the control group, and L of FIG. 11 shows a result of confirming the expression of iNOS in the control group. In FIG. 11, ** means P>0.01, *** means P>0.001, # means P>0.05, ## means P>0.01, and ### means P>0.001.

FIG. 12 shows results of analyzing the mRNA expression of inflammatory factors by RT-PCR by administering P-siSTAT3 of the present disclosure for each concentration to an animal model of autoimmune neurological disease. A of FIG. 12 shows a result of confirming an inhibitory effect of P-siSTAT3 for each concentration on the expression of each factor, B of FIG. 12 shows a result of confirming the expression of COX-2, C of FIG. 12 shows a result of confirming the expression of iNOS, D of FIG. 12 shows a result of confirming the expression of IL-1β, E of FIG. 12 shows a result of confirming the expression of IL-6, F of FIG. 12 shows a result of confirming the expression of TNF-α, G of FIG. 12 shows a result of confirming the expression of MIP-1α, and H of FIG. 12 shows a result of confirming the expression of RANTES. In FIG. 12, * means P>0.05, ** means P>0.01, *** means P>0.001, ## means P>0.01, and ### means P>0.001.

FIG. 13 shows results of analyzing effects of P-siSTAT3 of the present disclosure on immune cell invasion and microglial activation by flow cytometry by administering P-siSTAT3 of the present disclosure for each concentration to an animal model of autoimmune neurological disease. A of FIG. 13 shows a result of confirming the expression of CD4 positive cells, B of FIG. 13 shows a result of confirming the expression of CD4+INF-γ positive cells, C of FIG. 13 shows a result of confirming the expression of CD4+IL-17A positive cells, D of FIG. 13 shows a result of confirming the expression of CD11b+CD45^{low} positive cells, and E of FIG. 13 shows a result of confirming the expression of CD11b+CD45^{high} positive cells.

### MODES

Hereinafter, examples of the present disclosure will be described in detail with reference to the accompanying drawings. In the following description, detailed descriptions of techniques well-known to those skilled in the art may be omitted. Further, in describing the present disclosure, the detailed description of associated known functions or constitutions will be omitted if it is determined to unnecessarily make the gist of the present disclosure unclear. In addition, terminologies used in the present disclosure are terminologies used to properly express preferred examples of the present disclosure, which may vary according to a user, an operator's intention, or customs in the art to which the present disclosure pertains.

Accordingly, definitions of the terminologies need to be described based on contents throughout this specification. Throughout this specification, unless explicitly described to the contrary, when a certain part “comprises” a certain component, it will be understood to imply the inclusion of stated elements, not the exclusion of any other elements.

The present disclosure provides a pharmaceutical composition for prevention or treatment of autoimmune neurological disorder, including nanoparticles consisting of STAT3-specific siRNA and a biodegradable polymer material.

As used in the present disclosure, the term “prevention” refers to any action that suppresses the symptoms of a specific disease or delays its progression by administering the composition of the present disclosure.

As used in the present disclosure, the term “treatment” refers to any action that improves or beneficially changes the symptoms of a specific disease by administering the composition of the present disclosure.

Small RNA (hereinafter referred to as “sRNA”) refers to ribonucleic acid with a length of about 17 to 25 nucleotides (hereinafter referred to as “nt”), that serves to regulate gene expression in vivo. The sRNA is largely classified into microRNA (hereinafter referred to as “miRNA”) and small interfering RNA (hereinafter referred to as “siRNA”) according to a production method. The miRNA is generated from hairpin RNA that partially forms a double helix, and the siRNA is derived from long double-strand RNA (hereinafter referred to as “dsRNA”). In general, sRNA, which plays an important role in various regulatory processes in the body, is classified as miRNA, and sRNA, which is used experimentally to control the expression of a specific gene, is classified as siRNA. The small interfering RNA (siRNA) refers to a short double-stranded RNA that may induce RNA interference by cleaving specific mRNA. The siRNA is not limited to form complete pairing of a double-stranded RNA portion paring RNAs, but may include a portion which is not paired by mismatch (corresponding bases are not complementary), bulge (there is no corresponding base on one chain), etc. The length of the pairing bases may preferably be 50 to 80 bases. An end structure of siRNA may be both a blunt end and an overhang end as long as the end structure inhibits the expression of a target gene by an RNA interference effect. A sticky end structure may be both a 3′ end overhang structure or a 5′ end overhang structure.

According to one example of the present disclosure, the STAT3-specific siRNA may include at least one base sequence selected from the group consisting of SEQ ID NO: 1 and SEQ ID NO: 2, and may be siRNA that complementarily binds to a gene encoding a STAT3 protein. Preferably, the base sequence represented by SEQ ID NO: 1 may be a sense strand that complementarily binds to a STAT3 gene, and the base sequence represented by SEQ ID NO: 2 may be an antisense strand, which is a complementary sequence thereto.

The STAT3-specific siRNA according to the present disclosure has a base sequence designed to complementarily bind to mRNA encoding the corresponding gene, and thus may effectively inhibit the expression of the corresponding gene, thereby significantly inhibiting the expression of the corresponding protein. In addition, the 3′ end of the siRNA may include an overhang, which is a structure including one or two or more unpaired nucleotides.

The base sequence represented by “SEQ ID NO: 1” or “SEQ ID NO: 2” of the present disclosure is a base sequence including uracil (U) as an RNA sequence, but according to the ST.26 indication standard of WIPO according to the international sequence cataloging, the indication of uracil is converted to thymine (T), and the base sequence represented by SEQ ID NO: 1 or SEQ ID NO: 2 of the present disclosure is indicated as a sequence including T like DNA, but may be substantially a sequence including U of RNA.

The present disclosure may also include the siRNA itself, but may also include a fragment thereof, but is not limited thereto.

In the present disclosure, variants of the base sequence represented by SEQ ID NO: 1 or SEQ ID NO: 2 are also included within the scope of the present disclosure. The present disclosure is a concept including equivalents of the base sequence, for example, variants in which some base sequences are modified by deletion, substitution or insertion, but may have the same functional effect as the siRNA as a pharmaceutical composition for prevention or treatment of autoimmune neurological disorder. Specifically, the siRNA may include base sequences having at least 70%, more preferably at least 80%, even more preferably at least 90%, most preferably at least 95% sequence homology with the base sequence represented by SEQ ID NO: 1 or SEQ ID NO: 2, respectively. The “% of sequence homology” with the RNA is confirmed by comparing two optimally aligned sequences with a comparison region, and a portion of an RNA sequence in the comparison region may include addition or deletion (i.e., gap) compared to a reference sequence (without addition or deletion) for an optimal alignment of the two sequences.

In the present disclosure, the siRNA may have a ribonucleic acid unit structure that exists in nature without modification, or may be chemically modified. These chemical modifications of siRNA are intended to enhance in vivo stability, impart resistance to nucleases, and reduce non-specific immune responses.

Through the chemical modifications, various properties of siRNA may be improved compared to unmodified siRNA, in which the various properties include increased resistance to nucleases, increased cellular uptake, improved cellular targeting, increased stability, decreased interferon activity, and reduced off-target effects such as immune responses and sense effects, without affecting the RNAi ability.

The method for chemical modification of the siRNA is not particularly limited, and those skilled in the art may synthesize and modify the siRNA in a desired manner using a method known in the art. For example, the method for chemical modification may be used in combination with at least one selected from a modification by substitution of an —OH group at a 2′ carbon position of a sugar structure in a nucleotide with —CH₃ (methyl), —OCH₃ (methoxy), —NH₂, —F (fluorine), —O-2-methoxyethyl-O-propyl, —O-2-methylthioethyl, —O-3-aminopropyl, —O-3-dimethylaminopropyl, —O—N-methylacetamido or —O-dimethylamidooxyethyl; a modification by substitution of oxygen in the sugar structure in a nucleotide with sulfur; or a modification of a nucleotide bond to a phosphorothioate, boranophosphate, or methyl phosphonate bond, and also used with modifications in the form of PNA (peptide nucleic acid), LNA (locked nucleic acid), or UNA (unlocked nucleic acid), and may be used to attach a ligand such as cholesterol, biotin, or a cell penetrating peptide to the 5′- or 3′-end of the siRNA.

Among the chemical modifications, the resistance to nucleic acid degradation by nuclease may be increased by a method of replacing the phosphodiester bonds of the siRNA sense and antisense strands with phosphorothioate or boranophosphate bonds. For example, the 3′-end phosphodiester bonds of both the sense and antisense strands of siRNA may be changed to phosphorothioate bonds.

Among the chemical modifications, there is a method of introducing ENA (ethylene bridge nucleic acid), PNA (peptide nucleic acid), LNA (locked nucleic acid), or UNA (unlocked nucleic acid) to the 5′ end, 3′ end, or both ends of the siRNA sense or antisense strand, thereby increasing siRNA stability and reducing immune responses and non-specific inhibitory effects without affecting RNAi ability.

In addition to the chemical modifications, various chemical modifications may be applied, and these chemical modifications may be performed in only one form of modification or in combination with other chemical modifications.

However, in the modifications, it is preferable to perform the minimal modification, so as not to reduce the gene expression inhibition activity while stabilizing the siRNA double-stranded structure.

In the present disclosure, the biodegradable polymer material may be at least one selected from the group consisting of poly(lactic-co-glycolic acid) (PLGA), poly-L-lactic acid, poly-glycol acid, poly-D-lactic acid-co-glycol acid, poly-L-lactic acid-co-glycol acid, poly-D,L-lactic acid-co-glycol acid, polycaprolactone, poly-valerolacton, poly-hydroxy butyrate, poly-hydroxy valerate, dextran, poly-phosphazen, and poly-amino ester. More preferably, the biodegradable polymer material may be PLGA.

The PLGA is a biodegradable and biocompatible copolymer. The PLGA is synthesized from a ring-opening copolymer of 1,4-dioxane-2,5-diones, which is a cyclic dimer of two different monomers of glycolic acid and lactic acid. The PLGA is an FDA-approved drug carrier that has been applied in therapeutic fields by carrying various drugs, and a technology for manufacturing biodegradable polymer nano/microparticles has recently attracted much attention through research on not only the use as a delivery system, but also vaccine manufacture, bioimmune disease treatment, and the like.

In the present disclosure, the STAT3-specific siRNA is not limited thereto, but may bind to the surface of the biodegradable polymer material by simple covalent bonding, ionic bonding, linker-mediated covalent bonding, or the like. The linker mediating the covalent bonding is covalently bonded to the biodegradable polymer material at the end of the STAT3-specific siRNA, and is not particularly limited as long as providing bonds that may be degraded in a specific environment as needed. Accordingly, the linker may be used with any compound that binds to activate the STAT3-specific siRNA and/or the biodegradable polymer material during the preparing process of the nanoparticle according to the present disclosure without limitation.

According to one example of the present disclosure, it was confirmed that when the nanoparticles consisting of the STAT3-specific siRNA and the biodegradable polymer material according to the present disclosure were administered to an experimental autoimmune encephalomyelitis (EAE) animal model to inhibit demyelination of the spinal cord. In addition, the nanoparticles consisting of the STAT3-specific siRNA and the biodegradable polymer material according to the present disclosure inhibited the phosphorylation of STAT3, reduced the expression of Iba-1 or GFAP, and effectively inhibited the expression of inflammatory cytokines, inflammation-mediating enzymes, or chemokines. More specifically, the nanoparticles consisting of the STAT3-specific siRNA and the biodegradable polymer material according to the present disclosure inhibited the expression of at least one inflammatory cytokine selected from the group consisting of IL-1β, IL-6, and TNF-α. In addition, the nanoparticles consisting of the STAT3-specific siRNA and the biodegradable polymer material according to the present disclosure inhibited the expression of at least one inflammation-mediating enzyme selected from the group consisting of COX-2 and iNOS. In addition, the nanoparticles consisting of the STAT3-specific siRNA and the biodegradable polymer material according to the present disclosure inhibited the expression of at least one chemokine selected from the group consisting of MIP-1α and RANTES. In addition, the nanoparticles consisting of the STAT3-specific siRNA and the biodegradable polymer material according to the present disclosure showed an effect of reducing the invasion or activity of immune cells. More specifically, the nanoparticles consisting of the STAT3-specific siRNA and the biodegradable polymer material according to the present disclosure reduced the invasion or activity of at least one selected from the group consisting of Th1 and Th17. Furthermore, it was confirmed that the nanoparticles consisting of the STAT3-specific siRNA and the biodegradable polymer material according to the present disclosure may prevent damage to a blood-brain barrier (BBB) in the spinal cord.

Accordingly, the nanoparticles consisting of the STAT3-specific siRNA and the biodegradable polymer material according to the present disclosure may effectively prevent, alleviate or treat autoimmune neurological disorder. The autoimmune neurological disorder may be at least one selected from the group consisting of multiple sclerosis, neuromyelitis optica, acute disseminated encephalomyelitis, ascending myelitis, central myelitis, descending myelitis, transverse myelitis, myasthenia gravis, guillain-barre syndrome, autoimmune uveitis, autoimmune encephalopathy and chronic inflammatory demyelinating polyneuropathy, but is not limited thereto. Preferably, the autoimmune neurological disorder may be at least one selected from the group consisting of multiple sclerosis and encephalomyelitis, but is not limited thereto.

The pharmaceutical composition of the present disclosure may further include an adjuvant in addition to the active ingredient. The adjuvant may be used with any adjuvant known in the art without limitation, but further include, for example, a Freund's complete adjuvant or an incomplete adjuvant to increase the effect thereof.

The pharmaceutical composition according to the present disclosure may be prepared in the form of incorporating the active ingredient into a pharmaceutically acceptable carrier. Here, the pharmaceutically acceptable carrier includes carriers, excipients and diluents commonly used in a pharmaceutical field. The pharmaceutically acceptable carrier that may be used in the pharmaceutical composition of the present disclosure is not limited thereto, but may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil.

The pharmaceutical composition according to the present disclosure may be formulated and used in the form of oral formulations such as powders, granules, tablets, capsules, suspensions, emulsions, syrups, aerosols, etc., external preparations, suppositories, and sterile injectable solutions according to a conventional method.

The formulations may be prepared by using diluents or excipients, such as a filler, an extender, a binder, a wetting agent, a disintegrating agent, a surfactant, etc., which are generally used. Solid formulations for oral administration include tablets, pills, powders, granules, capsules, etc., and these solid formulations may be prepared by mixing at least one or more excipients, for example, starch, calcium carbonate, sucrose, lactose, gelatin, etc. with the active ingredient. Further, lubricants such as magnesium stearate and talc may be used in addition to simple excipients. Liquid formulations for oral administration may correspond to suspensions, oral liquids, emulsions, syrups, etc., and may include various excipients, for example, a wetting agent, a sweetener, an aromatic agent, a preserving agent, etc., in addition to the commonly used diluents, such as water and liquid paraffin. Formulations for parenteral administration include sterile aqueous solutions, non-aqueous solvents, suspensions, emulsions, lyophilized agents, and suppositories. As the non-aqueous solvent and the suspension, propylene glycol, polyethylene glycol, vegetable oil such as olive oil, injectable ester such as ethyl oleate, etc. may be used. As the base material of the suppository, witepsol, Tween 61, cacao butter, laurinum, glycerogelatin, etc. may be used.

The pharmaceutical composition of the present disclosure may be administered to a subject through various routes. All methods of administration may be expected, and the pharmaceutical composition may be administered by, for example, oral, intravenous, intramuscular, subcutaneous, and intraperitoneal injection.

The dose of the pharmaceutical composition according to the present disclosure is selected in consideration of the age, body weight, sex, and physical condition of the subject. It is obvious that the concentration of the active ingredient included in the pharmaceutical composition may be variously selected according to a target, and preferably included in the pharmaceutical composition at a concentration of 0.01 to 5,000 μg/ml. When the concentration is less than 0.01 μg/ml, pharmaceutical activity may not be exhibited, and when the concentration is more than 5,000 μg/ml, toxicity to the human body may be exhibited. The administration may be performed once a day or several times a day. The dose does not limit the scope of the present disclosure in any aspect.

In addition, the pharmaceutical composition of the present disclosure may be used alone or in combination with surgery, radiation therapy, hormone therapy, chemotherapy, and methods of using biological response modifiers for treatment of autoimmune neurological disorder. In addition, the pharmaceutical composition of the present disclosure may further include any compound or natural extract that has already been verified as safe and has been known to have an effect of preventing or treating autoimmune neurological disorder, in order to increase or enhance the effect of preventing, alleviating, or treating autoimmune neurological disorder, in addition to the nanoparticles consisting of the STAT3-specific siRNA and the biodegradable polymer material according to the present disclosure.

Further, the present disclosure provides a food composition for prevention or alleviation of autoimmune neurological disorder, including nanoparticles consisting of STAT3-specific siRNA and a biodegradable polymer material.

Further, the present disclosure provides a healthy functional food composition for prevention or alleviation of autoimmune neurological disorder, including nanoparticles consisting of STAT3-specific siRNA and a biodegradable polymer material.

The food composition according to the present disclosure includes all forms, such as functional food, nutritional supplements, health food, health supplements, and food additives. The type of food composition may be formulated in any one form selected from the group consisting of powders, tablets, capsules, pills, and liquids according to a conventional method known in the art, but is not limited thereto. The food composition may be prepared in various forms using methods known in the art.

For example, as the health food, the nanoparticles themselves consisting of the STAT3-specific siRNA and the biodegradable polymer material of the present disclosure may be ingested by granulation, encapsulation and powdering or prepared and drunk in the form of tea, juice, and drinks. In addition, the nanoparticles consisting of the STAT3-specific siRNA and the biodegradable polymer material of the present disclosure may be mixed with a material or active ingredient known to have the prevention, alleviation, or treatment activity of autoimmune neurological disorder to be prepared in the form of a composition.

In addition, the functional food may be prepared by adding the nanoparticles consisting of the STAT3-specific siRNA and the biodegradable polymer material of the present disclosure to beverages (including alcoholic beverages), fruits and processed foods thereof (e.g., canned fruit, bottled food, jam, marmalade, etc.), fish, meat and processed foods thereof (e.g., ham, sausage, corned beef, etc.), bread and noodles (e.g., udon, buckwheat noodles, ramen, spaghetti, macaroni, etc.), fruit juice, various drinks, cookies, taffy, dairy products (e.g., butter, cheese, etc.), edible vegetable oil, margarine, vegetable protein, retort foods, frozen foods, various seasonings (e.g., soybean paste, soy sauce, sauce, etc.), etc.

In addition, the food composition of the present disclosure may include conventional food additives, and the suitability as the “food additive” is determined by the specifications and standards for the corresponding item in accordance with the general rules of the Food Additive Codex, general test methods, and the like approved by the Food and Drug Administration, unless otherwise specified. The items disclosed in the “Food Additives Codex” may include, for example, chemical composites such as ketones, glycine, calcium citrate, nicotinic acid, cinnamic acid, etc., natural additives such as persimmon color, licorice extract, crystal cellulose, Kaoliang color, guar gum, etc., and mixed formulations such as sodium L-glutamic acid formulations, alkali agents for noodles, preservative formulations, tar color formulations, etc.

In the food composition of the present disclosure, the nanoparticles consisting of the STAT3-specific siRNA and the biodegradable polymer material of the present disclosure may be included preferably in an amount of 0.00001 to 50 wt % based on the food composition. When the content is less than 0.00001 wt %, the effect thereof is insignificant, and when the content is more than 50 wt %, an increase in effect compared to the amount used is minimal, which is uneconomical.

In addition, in order to use the nanoparticles consisting of the STAT3-specific siRNA and the biodegradable polymer material of the present disclosure in the form of food additives, the nanoparticles may be prepared and used in the form of tablets, capsules, powders, granules, liquids, pills, etc.

When the composition of the present disclosure is prepared as beverages, like general beverages, the composition may include various flavoring agents or natural carbohydrates as an additional ingredient. The above-mentioned natural carbohydrates may be used with monosaccharides such as glucose and fructose, disaccharides such as maltose and sucrose, natural sweeteners such as dextrin and cyclodextrin, synthetic sweeteners such as saccharin and aspartame, and the like. A ratio of the natural carbohydrates may be generally about 0.01 to 10 g, preferably about 0.01 to 0.1 g per 100 ml of the composition of the present disclosure.

In addition, the composition of the present disclosure may include various nutrients, vitamins, electrolytes, flavoring agents, coloring agents, pectic acid and salts thereof, alginic acid and salts thereof, organic acid, a protective colloidal thickener, a pH adjusting agent, a stabilizer, a preservative, glycerin, alcohols, a carbonic acid agent used in a carbonated drink, or the like. In addition, the composition of the present disclosure may include pulps for preparing natural fruit juices, fruit juice beverages or vegetable beverages. These ingredients may be used independently or in combination. Although the ratio of these additives is not greatly important, generally, the ratio thereof is selected in the range of 0.01 to 0.1 parts by weight per 100 parts by weight of the composition of the present disclosure.

In the present disclosure, the “health supplement food” or “health functional food” refers to food prepared and processed using raw materials or ingredients with functionality, which are useful for the human body according to the Health Functional Foods Act. The “functionality” means ingestion for the purpose of adjusting nutrients for the structures and functions of the human body or obtaining useful effects on health applications such as physiological actions.

Further, the present disclosure provides a method for preventing or treating autoimmune neurological disorder, including administering to a subject nanoparticles consisting of STAT3-specific siRNA and a biodegradable polymer material.

The subject is preferably mammals, including humans, and patients in need of treatment for autoimmune neurological disorder include all patients being treated, patients who have previously received treatment, and patients in need of treatment for diseases, and may also include patients who have undergone surgical procedures for autoimmune neurological disorder.

The autoimmune neurological disorder may be at least one selected from the group consisting of multiple sclerosis, neuromyelitis optica, acute disseminated encephalomyelitis, ascending myelitis, central myelitis, descending myelitis, transverse myelitis, myasthenia gravis, guillain-barre syndrome, autoimmune uveitis, autoimmune encephalopathy and chronic inflammatory demyelinating polyneuropathy, but is not limited thereto. Preferably, the autoimmune neurological disorder may be at least one selected from the group consisting of multiple sclerosis and encephalomyelitis, but is not limited thereto.

Hereinafter, the present disclosure will be described in more detail through Examples. These Examples are to explain the present disclosure in more detail, and it will be apparent to those skilled in the art that the scope of the present disclosure is not limited to these Examples.

### <Example 1> Preparation for Confirming Effect of PLGA-siSTAT3 on Alleviating Autoimmune Neurological Disorder

### <1-1> Preparation of Poly-Lactic-Co-Glycolic Acid (PLGA) Nanoparticles Containing siSTAT3 (P-siSTAT3)

PLGA-siSTAT3 nanoparticles (P-siSTAT3) loaded with STAT3 siRAN (siSTAT3) in PLGA of the present disclosure were prepared. Specifically, 800 μl of TE 7.5 (Sigma-Aldrich, St. Louis, MO, USA) buffer containing 20 μM of STAT3 siRNA (SEQ ID NO: 1, 5′-AAA CGT GAG CGA CTC AAA CTG CCC T-3′ and SEQ ID NO: 2, 5′-AGG GCA GTT TGA GUC GCT CAC GTT T-3′) and dichloromethane (DCM) containing 25 mg of PLGA (Corbion, Amsterdam, the Netherlands) were prepared. Meanwhile, in order to confirm the affinity of P-siSTAT3 of the present disclosure with microglia, PLGA containing Coumarin-6, which exhibited green fluorescence, was also used instead of STAT3 siRAN (siSTAT3). Thereafter, 200 μl of the TE7.5 buffer was added dropwise to dichloromethane to prepare a mixture. The mixture was emulsified into a primary W1/O emulsion by sonication (UP100H ultrasonic processor, Hielscher Ultrasonics GmbH, Teltow, Germany). 2 ml of 2% PVA1500 (w/v) was added to the primary W1/O emulsion and then sonicated to prepare a W1/O/W2 double emulsion. The prepared W1/O/W2 double emulsion was diluted in 6 ml of 2% PVA1500 (w/v) and stirred at room temperature for 3 hours to evaporate DCM. In addition, PLGA nanoparticles were collected by ultracentrifugation (Optima™ Max Ultracentrifuge, Beckman COULTER, Brea, CA) at 38,000 g for 10 minutes at 4° C. The collected PLGA nanoparticles were washed twice with deionized RNase-free water. The washed PLGA nanoparticles were re-diluted in water and then prepared by freeze-drying.

### <1-2> Preparation and Classification of Multiple Sclerosis or Encephalomyelitis Animal Model

In order to confirm an effect of PLGA-siSTAT3 of the present disclosure on alleviating multiple sclerosis or encephalomyelitis, an experimental autoimmune spondylitis animal model, which was an animal model of multiple sclerosis and encephalomyelitis, was prepared. Specifically, an experimental autoimmune encephalomyelitis (EAE) animal model was prepared using a myelin oligodendrocyte glycoprotein (MOG₃₅₋₅₅) peptide, and the animal model was stabilized for 1 week by preparing 8-week-old female C57BL/6 mice (Narabiotechnology, Korea; weighing 20 to 21 g) after birth. Thereafter, 100 μl of the emulsion was injected subcutaneously into both lumbar regions of the mice in an EAE group by mixing an emulsion containing 200 μg of an MOG₃₅₋₅₅ peptide (Sigma-Aldrich, USA) and an incomplete Freund's adjuvant (IFA; Difco, USA) containing 550 μg of Mycobacterium tuberculosis (Difco, USA). At the same time, 200 ng of pertussis toxin (PTX; List Biologic, USA) was injected intraperitoneally. After 48 hours, the same amount of PTX was additionally injected intraperitoneally. In a chronic EAE animal model induced by the MOG₃₅₋₅₅ peptide, a clinical symptom scale was divided into 8 stages and confirmed. That is, the clinical symptom scale was divided into no response (stage 0), tail tip drooping (stage 1), entire tail drooping (stage 2), mild paralysis of both hind legs (stage 3), paralysis of both hind legs (stage 4), paralysis up to one front leg (stage 5), paralysis up to both front legs (stage 6), and death (stage 7). The P-siSTAT of the present disclosure was dissolved in PBS at concentrations of 5, 10, and 20 mg/ml and administered once into the spinal cord at the time when clinical symptoms appeared (on days 7 to 8) after EAE induction, and P-siSTAT3 was adjusted to be 10 μl of a dose per subject. In addition, as a control group for P-siSTAT3 of the present disclosure, “scrambled siRNA” was RNA in which the base sequences of the siRNA were randomly mixed, and was used with siRNA specifically binding to target mRNA. Thereafter, for a behavioral experiment, mice were divided into groups shown in Table 1 below.

TABLE 1

| Experimental group | Drug administration |
|---|---|
| Sham group | Normal control group in which MOG₃₅₋₅₅ peptide and P- |
| | siSTAT3 were not administered |
| EAE + PBS group | Group in which EAE was induced with MOG₃₅₋₅₅ peptide |
| | and PBS was directly administered intrathecally |
| EAE + P-siSTAT3 group | Group in which EAE was induced with MOG₃₅₋₅₅ peptide |
| | and P-siSTAT3 was dissolved in PBS and directly |
| | administered intrathecally |
| EAE + PLGA-scrambled | Group in which EAE was induced with MOG₃₅₋₅₅ peptide |
| siRNA (P-Sc) group | and P-Sc was administered intrathecally |
| EAE + siRNA (siSTAT3) | Group in which EAE was induced with MOG₃₅₋₅₅ peptide |
| group | and siRNA was directly administered intrathecally |
| P-siSTAT3 group | Group in which P-siSTAT3 was dissolved in PBS (20 |
| | mg/ml) and directly administered intrathecally in normal |
| | mice in which EAE was not induced with MOG₃₅₋₅₅ |
| | peptide |

In addition, for histological and molecular biological analyses, mice were classified into groups disclosed in Table 2 below.

TABLE 2

| Experimental group | Drug administration |
|---|---|
| Sham group | Normal control group in which MOG₃₅₋₅₅ peptide and P- |
| | siSTAT3 were not administered |
| EAE + PBS group | Group in which EAE was induced with MOG₃₅₋₅₅ peptide |
| | and PBS was directly administered intrathecally |
| EAE + P-siSTAT3 group | Group in which EAE was induced with MOG₃₅₋₅₅ peptide |
| | and P-siSTAT3 was dissolved in PBS and directly |
| | administered intrathecally |
| EAE + PLGA-STAT3 | Group in which EAE was induced with MOG₃₅₋₅₅ peptide |
| scrambled siRNA (P-Sc) group | and P-Sc was administered intrathecally |
| P-siSTAT3 group | Group in which P-siSTAT3 was dissolved in PBS (20 |
| | mg/ml) and directly administered intrathecally in normal |
| | mice in which EAE was not induced with MOG₃₅₋₅₅ |
| | peptide |

### <1-3> Histopathological Analysis

For histopathological analysis, on days 16 to 17 when clinical symptoms worsened after EAE was induced in mice of each group of Examples 1-2, the mice were anesthetized, fixed by cardiac perfusion with 4% paraformaldehyde (PFA), and the spinal cord in the lumbar region (lumbar 4-5; L4-5) was extracted. The extracted spinal cord was immersed in 4% PFA and additionally fixed at 4° C. for one day, and then immersed at 4° C. for 3 days by replacement with 0% sucrose to prevent freezing damage.

Thereafter, in order to confirm an inhibitory effect of P-siSTAT3 on demyelination in an EAE animal model, the degree of demyelination was confirmed using luxol fast blue (LFB) staining, which stained phospholipids, a main component of myelin sheath, and the invasion of immune cells was confirmed using H&E staining. Specifically, for LFB staining, the spinal cord was cut at a 10 μm thickness using a cryostat to prepare sections, and then the sections were immersed in 100% xylene in the order of 100%, 95%, 90%, 80%, and 70% EtOH for 2 minutes each, and then stained with 0.1% Luxol Fast Blue Solution A and destained with Solution B. Thereafter, the sections were immersed and dehydrated in 70%, 80%, 90%, 95%, and 100% EtOH solutions for 2 minutes each, and then immersed in 100% xylene and then treated with a mounting solution. Since axons existed in the white matter of the outer part of the spinal cord, when normal tissue was stained, the white matter of the spinal cord appeared dark blue. However, when the myelin sheaths were lost due to demyelination after trauma, the staining intensity became weaker and had blue, and thus the degree of demyelination was confirmed.

For H&E staining, spinal cord tissue prepared in the same manner as above was placed on a slide for H&E staining, immersed in a hematoxylin solution, left for 3 minutes, and then washed with distilled water three times for 1 minute each. Then, the spinal cord tissue was immersed in an eosin solution for 10 minutes, washed with distilled water three times for 1 minute each, and immersed and dehydrated sequentially in 70%, 80%, 90%, 95%, and 100% EtOH for 2 minutes, and then reacted with xylene to confirm the invasion of immune cells.

### <1-4> Immunohistochemical Staining

For immunohistochemical staining, frozen sections were prepared using the same method as in Example 1-3. Thereafter, the sections were placed in a plate containing a blocking solution (10 ml prepared by mixing 200 μg bovine serum albumin (BSA, Bovine serum albumin, Sigma, USA), 500 μl Fetal bovine serum (FBS, Gibco, Germany), 500 μl Normal goat serum (NGS, Vector, USA), and 10 μl Triton X-100 (Sigma, USA) in PBS) and reacted for 1 hour, then washed with PBS, and reacted for 12 hours or more by replacement with a solution of a primary antibody against Iba-1 (ionized calcium-binding adapter molecule-1) as a marker of microglia, a primary antibody against MBP (Myelin basic protein) as a marker of myelin sheath, a primary antibody against GFAP (Glial fibrillary acidic protein) as a marker of astrocytes, or a primary antibody against Olig-2 as a marker of oligodendrocytes diluted in the blocking solution. Thereafter, the sections were washed with PBS, reacted for 1 hour by replacement with a solution of a secondary antibody diluted 1:1000 in PBS, and then washed with PBS. Thereafter, a cover glass was covered with a mounting solution.

### <1-5> Western Blot Analysis

To confirm the inhibitory effects of P-siSTAT3 on demyelination, glial activation, secretion of inflammatory cytokines, and activation of inflammatory mechanisms in an animal model of EAE induced by a MOG₃₅₋₅₅ peptide, the expression of various markers was confirmed using Western blot analysis. Specifically, in mice of each group, the mice were humanely sacrificed between days 16 and 17, when clinical symptoms were worsened, and then the lumbar spine (L4-5) of the lumbar region was extracted, added with 200 μl of a protein lysis buffer (10 mM Tris, 0.5 mM EDTA, 0.25 M sucrose), and then proteins were separated using a grinder. The separated proteins were quantified for each subject using a Bradford (Bio-Rad, USA) method using bovine serum albumin (BSA, Sigma, USA), and then 25 μg of proteins was separated by 10% Sodium Dodecyl Sulfate-polyacrylamide gel electrophoresis (SDS-PAGE) and transferred to a polyvinylidene difluoride (PVDF) membrane (Gendepot, UK). The PVDF membrane was blocked with 5% bovine serum albumin (BSA, Sigma, USA) for 1 hour. The PVDF membrane was washed with TBST, and then reacted at 4° C. for one day with primary antibodies [STAT3 (inflammatory marker), GFAP (astrocyte marker), iNOS (inflammatory marker), and GAPDH] diluted 1:1,000 in 3% BSA, washed three times for 10 minutes each with TBST, and then reacted with secondary antibodies at room temperature for 1 hour. After the secondary antibody reaction, the PVDF membrane was washed with TBST and then the bands were confirmed using an ECL system (Santacruz, USA). The identification and quantitative analysis of the proteins were performed using imaging equipment ChemiDoc XRS+ (Bio-Rad).

### <1-6> Reverse Transcription Polymerase Chain Reaction (RT-PCR)

To confirm the inhibitory effect of P-siSTAT3 on the secretion of inflammatory cytokines and activation of inflammatory mechanisms in an animal model of EAE induced by a MOG₃₅₋₅₅ peptide, mRNA expression was confirmed using real-time PCR analysis. Specifically, in mice of each group, the mice were humanely sacrificed between days 16 and 17, when clinical symptoms were worsened, and then the spinal cord (lumbar spine 4-5; L4-5) in the lumbar region was extracted. The extracted spinal cord tissue was ground with a grinder by adding 1 ml of Trizol (Invitrogen, USA) and centrifuged to isolate RNA. To synthesize cDNA, 1 μg of total RNA was reacted at 37° C. for 1 hour in a reaction mixture containing 0.5 μg of Oligo dT, 0.5 mM dNTP mix, 5× first-strand buffer, RNase out, 5 mM dithiothreitol (DTT), and M-MLV reverse transcriptase. RT-PCR analysis was performed according to the manufacturer's manual (RT-PCR kit; Roche, Germany), and primer sequences used for PCR were shown in Table 3 below. For PCR amplification, specific oligonucleotide primer pairs were reacted with 2 μl of cDNA and 0.6 U of Econo TaqDNA polymerase in 6 μl of PCR Master Mix (Lucigen, WI, USA). 7.5 μl of the PCR product was electrophoresed on a 3% agarose gel and the mRNA expression was confirmed by staining with ethidium bromide in a Trend Illuminator, and the expression level of each gene was quantified with glyceraldehyde 3-phosphate dehydrogenase (GAPDH).

TABLE 3

| Target | Primer | | SEQ |
|---|---|---|---|
| gene | Sequence (5′ - 3′) | | ID: |
| COX-2 | COX-2_F | GCT CGG CTT CCA GTA | 3 |
| | | TTG AG | |
| | COX-2_R | AGA AGG AAA TGG CTG | 4 |
| | | CAG AA | |
| iNOS | iNOS_F | GGC AAA CCC AAG GTC | 5 |
| | | TAG GTT | |
| | iNOS_R | TCG CTC AAG TTC AGC | 6 |
| | | TTG GT | |
| IL-1β | IL-1β_F | TTG TTG CTG TGG AGA | 7 |
| | | AGC TGT | |
| | IL-1β_R | AAC GTC ACA CAC CAG | 8 |
| | | CAG GTT | |
| IL-6 | IL-6_F | TCC ATC CAG TTG CCT | 9 |
| | | TCT TGG | |
| | IL-6_R | CCA CGA TTT CCC AGA | 10 |
| | | GAA CAT | |
| TNF-α | TNF-α_F | AGC CCC CAG TCT GTA | 11 |
| | | TCC TT | |
| | TNF-α_R | CTC CCT TTG CAG AAC | 12 |
| | | TCA GG | |
| MIP-1α | MIP-1α_F | CAG CCA GGT GTC ATT | 13 |
| | | TTC CT | |
| | MIP-1α_R | AGG CAT TCA GTT CCA | 14 |
| | | GGT CA | |
| RANTES | RANTES_F | ACA CCA CTC CCT GCT | 15 |
| | | GCT TT | |
| | RANTES_R | GAC TGC AAG ATT GGA | 16 |
| | | GCA CTT G | |
| GAPDH | GAPDH_F | AGG TCA TCC CAG AGC | 17 |
| | | TGA ACG | |
| | GAPDH_R | CAC CCT GTT GCT GTA | 18 |
| | | GCC GTA T | |

### <1-7> Cell Line Culture

BV2 as a microglial cell line and bEND.3 as a brain tissue-derived epithelial cell line were maintained in DMEM supplemented with 10% FBS and 1% penicillin/streptomycin at 37° C. in a 5% CO₂ incubator. The cells were stabilized, and then seeded in 6-well plates at a density of 3×10⁵ cells/well and attached to the plate for 24 hours. Then, BV2 cells were treated with 1 μg/ml of LPS and stimulated for 4 hours. To investigate the effect of P-siSTAT3 on microglial activation and BBB damage, P-siSTAT3 (10, 50, and 100 μg/ml) was treated 1 hour before LPS stimulation. Next, a portion of the culture medium was obtained and cultured for 48 hours. After 48 hours, the pre-cultured bEND.3 cell line was treated with 2 ml of the BV2 culture medium, cultured for 48 hours, and the cells were collected for the following analysis.

### <1-8> Statistical Analysis

All results were analyzed using the SPSS 23.0 package (SPSS Inc, Chicago, USA), and the experimental results were represented as mean±standard deviation values. Statistical significance was verified using one-way analysis of variance (ANOVA) and then post-analyzed using Tukey post hoc, and when P-values were less than p<0.05 and p<0.01, it was considered statistically significant.

### <Example 2> Confirmation of Effect of P-siSTAT3 on Microglia

### <2-1> Confirmation of Effect of Microglia on Inflammatory Response

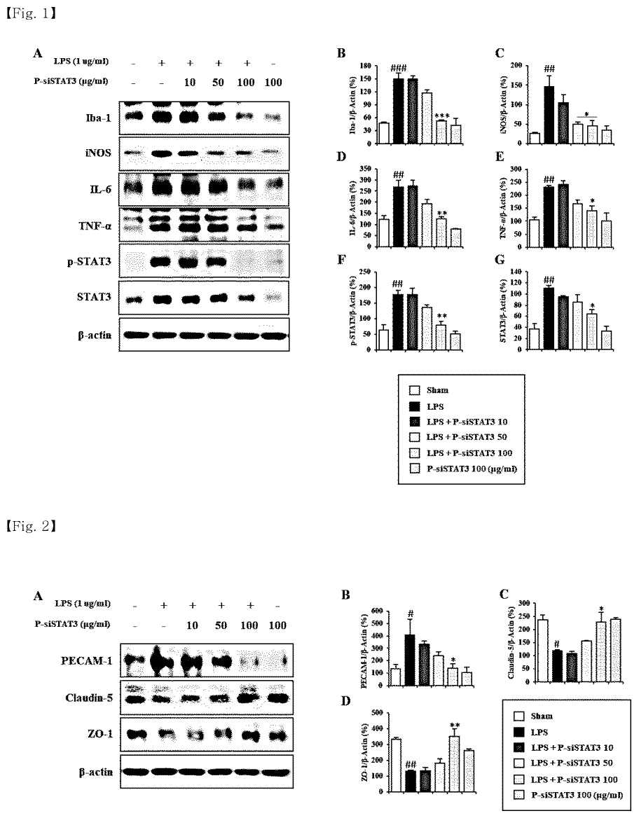

In animal models of multiple sclerosis (MS) and EAE, activation of microglia during inflammation and demyelination induced release of cytotoxic factors such as NOS and ROS to induce myelin damage, and triggered the release of proinflammatory cytokines IL-6 and TNF-α. Ultimately, this induced damage to the blood-brain barrier (BBB). Moreover, activated microglia inhibited the expression of rigid junction molecules (cadherin, occludin, and claudin-5), induced the death of oligodendrocyte, and increased BBB permeability. Therefore, in order to investigate whether P-siSTAT3 according to the present disclosure affected the inflammatory response of microglia, P-siSTAT3 was treated on a microglial cell line (BV2) and its effect was confirmed. As a result, it was confirmed that an experimental group that was treated with lipopolysaccharide (LPS) to induce an inflammatory response showed a significant increase in Iba-1 protein expression compared to a Sham experimental group. However, it was confirmed that in an experimental group treated with P-siSTAT3 according to the present disclosure, a dose-dependent increase in Iba-1 protein expression was inhibited (A and B of FIG. 1). Consistent with these results, the protein expression of representative inflammatory enzyme (iNOS), cytokines (IL-6, TNF-α), and major signaling pathways (p-STAT3 and STAT3) were significantly increased in an LPS-stimulated group compared to the sham experimental group. In contrast, in the P-siSTAT3 treated group according to the present disclosure, it was confirmed that the expression thereof was decreased in a dose-dependent manner (A and C to G of FIG. 1). Through these results, it was confirmed that the P-siSTAT3 of the present disclosure may inhibit the inflammatory response of microglia.

### <2-2> Confirmation of Inhibitory Effect of Microglia on Inflammatory Response and BBB Damage

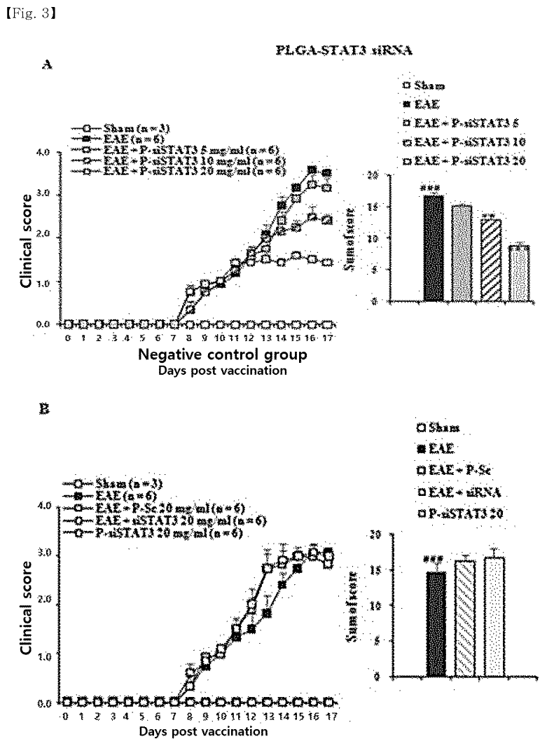

In addition, the role of P-siSTAT3 was analyzed under conditions of activated microglia-mediated BBB damage. To investigate the effect of microglia whose activation was inhibited by P-siSTAT3 on BBB damage, 2 ml of a culture medium (LPS-BCM) of BV2 cells induced by LPS and 2 ml of a culture medium (P-siSTAT3-BCM) of BV2 cells treated with P-siSTAT3 were treated to bEnd.3 cells. As a result, an experimental group treated with LPS-BCM showed higher expression of PECAM-1 protein, a major component molecule of BBB, and lower expression of claudin-5 and ZO-1 proteins, representative tight junction molecules, compared to an experimental group treated with PBS-BCM. In contrast, the experimental group treated with P-siSTAT3-BCM showed a decrease in PECAM-1 protein expression compared to the experimental group treated with LPS-BCM, and an improvement in the protein levels of claudin-5 and ZO-1 was observed (A to D of FIG. 2).

### <Example 3> Confirmation of Effect of P-siSTAT3 on Alleviating Multiple Sclerosis or Encephalomyelitis

### <3-1> Confirmation of Behavioral Improvement and Neuroprotective Effects of P-siSTAT3

It was confirmed whether PLGA-siSTAT3 of the present disclosure protected behavioral abnormalities and neurological damage caused by multiple sclerosis or encephalomyelitis. In each group of mice in Example 1-2, the clinical symptom scale was evaluated daily for 17 days from the day when EAE was induced. Behavioral symptoms in the EAE group began to appear on day 7 after EAE induction and reached a peak (3.6) on day 16, and the sum of clinical symptom indices from day 12 to day 17 after EAE induction was 16.7. However, in an EAE+P-siSTAT3 group, a decrease in the index of the clinical symptom scale was confirmed compared to the EAE group, and in the EAE+P-siSTAT3 group, a significant decrease in the index of the clinical symptom scale was confirmed in the groups treated with 10 and 20 mg/ml of P-siSTAT3. From day 12 to day 17 after EAE induction, the sum of clinical symptom indices was 13±0.8 in the group treated with 10 mg/ml of P-siSTAT3 and 8.8±0.6 in the group treated with 20 mg/ml of P-siSTAT3 (A of FIG. 3). In a P-Sc group and a siSTAT3 group, behavioral abnormalities were not significantly improved, and thus it was confirmed that only PLGA-siSTAT3 of the present disclosure may improve behavioral abnormalities (B of FIG. 3).

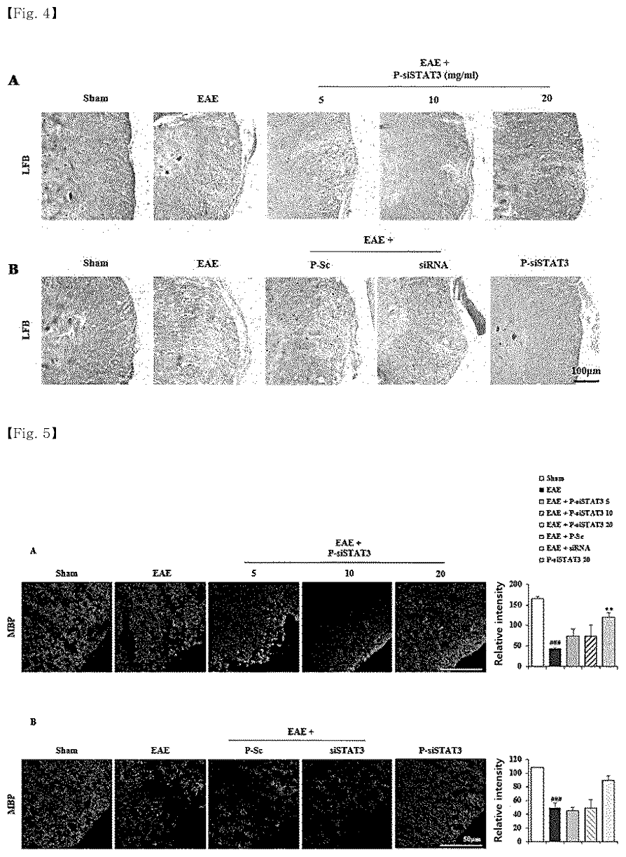

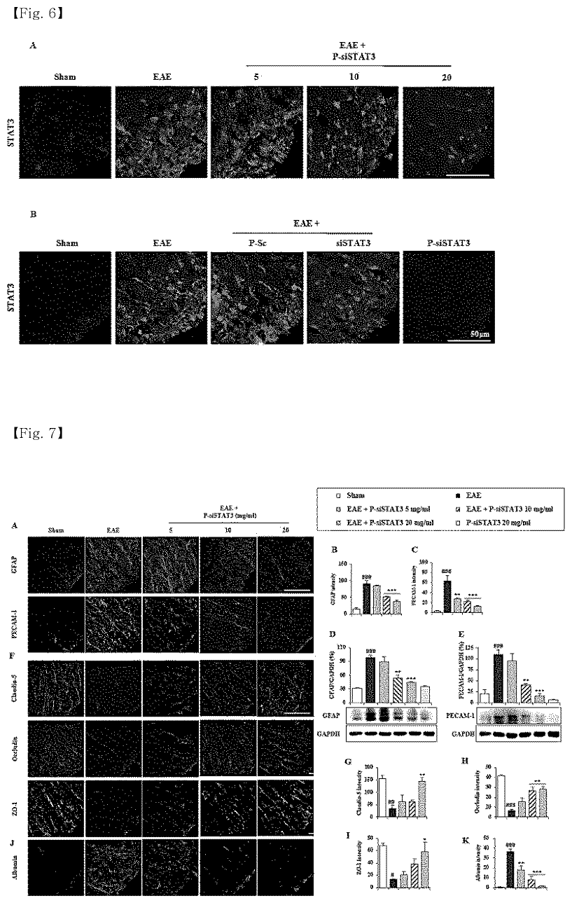

In addition, in order to confirm demyelination, LFB staining was performed, and as a result, it was confirmed that compared to the Sham group, demyelination was increased in the EAE group, but the increased demyelination was significantly reduced in the EAE+P-siSTAT3 group (FIG. 4 and A of FIG. 5). In addition, compared to the EAE group, in the P-Sc group and the siSTAT3 group, the demyelination was not significantly improved, and thus it was confirmed that the P-siSTAT3 of the present disclosure may improve EAE (B of FIG. 5).

### <3-2> Confirmation of STAT3 Signaling Inhibition by P-siSTAT3

It was confirmed whether the PLGA-siSTAT3 of the present disclosure blocked STAT3 signaling, and inhibition of signaling of related pathways was confirmed by immunostaining using an antibody against STAT3. As a result, as shown in A of FIG. 6, it was confirmed that the expression of STAT3, which was increased in the EAE group, was decreased in a concentration-dependent manner in the EAE+P-siSTAT3 group, and thus it was confirmed that inhibition of STAT3, an inflammatory signaling pathway in the spinal cord, reduced demyelination. In addition, no significant inhibition of STAT3 signaling was observed in the P-Sc group and the siSTAT3 group (B of FIG. 6), and thus it was confirmed that P-siSTAT3 of the present disclosure may inhibit STAT3 signaling.

### <3-3> Confirmation of BBB Damage Inhibition Effect of P-siSTAT3

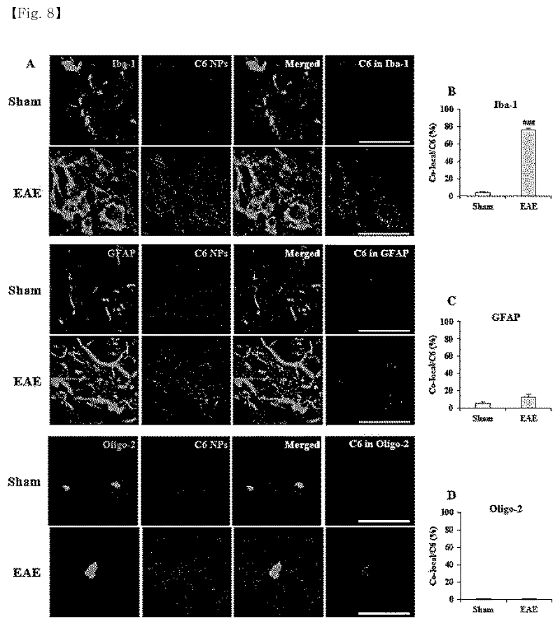

The BBB consisted of endothelial cells, pericytes, and astrocytes of the blood vessels of the central nervous system. The BBB damage was a major pathologic feature of multiple sclerosis. Accordingly, in the EVE animal model, the effect of P-siSTAT3 on the degree of BBB damage was confirmed through immunostaining and Western blot analysis between days 16 and 17, when clinical symptoms were most severe. As a result, the protein expression levels of GFAP as an activation marker of astrocytes and PECAM-1 as a cell adhesion protein were significantly increased in the EAE experimental group compared to the Sham experimental group. In contrast, in the P-siSTAT3 treated experimental group, the expression levels of the two markers were decreased in a dose-dependent manner (A to E of FIG. 7). The protein expression levels of junctional proteins, claudin-5, occludin, and ZO-1 (zonula occludens), were significantly reduced in the EAE group compared to the Sham group. In contrast, the expression levels of these three proteins increased by P-siSTAT3 treatment (F to I of FIG. 7). At the same time, as a result of evaluating the efficacy of P-siSTAT3 on the degree of albumin leaked into neural tissues in blood vessels due to BBB damage, it was confirmed that the expression level of albumin in the EAE group was increased compared to the Sham experimental group. However, the expression level of albumin was significantly reduced in the P-siSTAT3 treated group (J to K of FIG. 7). Through these results, it was clearly confirmed that P-siSTAT3 may prevent BBB damage in the spinal cord (lumbar cord) of an EAE animal model.

### <3-4> Confirmation of Selectivity of P-siSTAT3 for Microglia

Considering the neuroprotective effects of P-siSTAT3 on demyelination, neuroinflammation, and BBB damage, there was a need to differentiate types of cells targeted by P-siSTAT3. Therefore, the types of cells were differentiated using P-coumarin-6 included in P-siSTAT3. The siSTAT3 had no fluorescence, but coumarin-6 had green fluorescence, and thus cells expressing P-coumarin-6 were selected as cells targeted by P-siSTAT3.

As a result, high affinity of PLGA for microglia was observed in the spinal cord tissue of the EAE model. Specifically, immunostaining for cell type-specific markers, including Iba-1 (microglia marker), GFAP (astrocyte marker), and Oligo-2 (oligodendrocyte marker), was performed in the spinal cord tissue of the EAE model. As a result, the expression of P-coumarin-6 was clearly observed in microglia expressing Iba-1 (A to D of FIG. 8). Therefore, it was confirmed that P-siSTAT3 according to the present disclosure may selectively target microglia in the spinal cord tissue of the EAE model.

### <3-5> Confirmation of Spinal Cord Damage Protection of P-siSTAT3

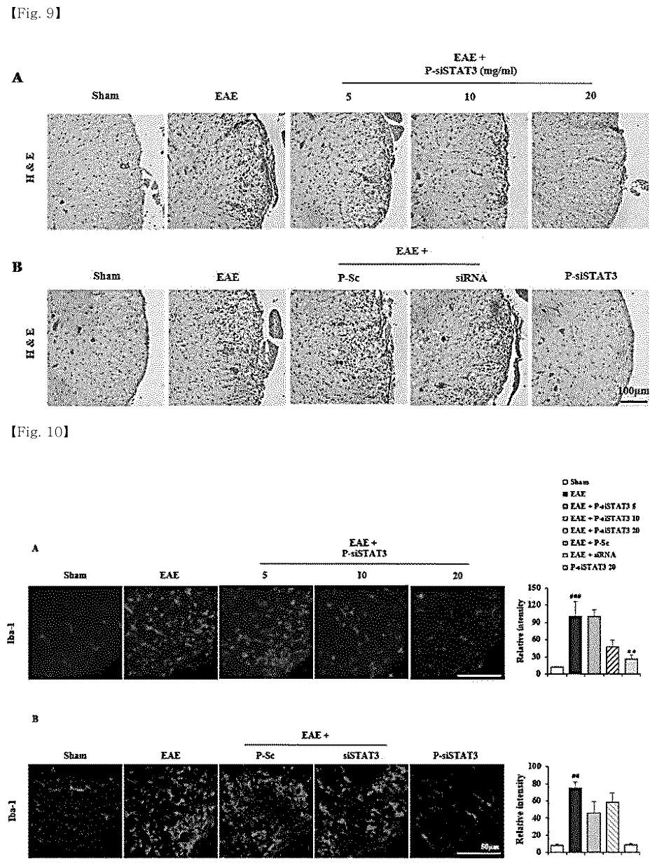

To determine whether PLGA-siSTAT3 protected damage to spinal cord tissue in an EAE animal model, histological damage was confirmed by H&E staining. As a result, as shown in FIG. 9, damage to spinal cord tissue was confirmed in the EAE group compared to the Sham group, but it was confirmed that tissue damage was improved in the P-siSTAT3 group compared to the EAE group. In addition, in the P-Sc group and the siRNA group, the spinal cord tissue damage was similar to that of the EAE group, and spinal cord damage was not improved, and thus it was confirmed that only P-siSTAT3 of the present disclosure may protect the spinal cord damage.

### <3-6> Confirmation of Immune Cell Invasion and Inflammation Inhibition of P-siSTAT3

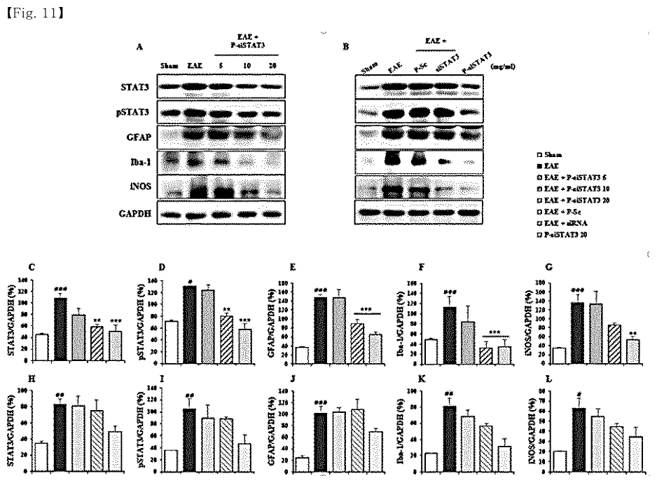

In order to confirm whether PLGA-siSTAT3 inhibited immune cell invasion and inflammatory response in an EAE animal model, immunohistochemical staining was used for Iba-1, a microglial activating factor. As a result, as shown in FIG. 10, it was confirmed that the EAE group showed increased invasion of immune cells in the spinal cord tissue and increased activation of microglia compared to the Sham group, but in the P-siSTAT3 group, invasion and activation of immune cells were reduced compared to the EAE group (A of FIG. 10). In the P-Sc group and the siRNA group, the invasion and activation of immune cells were not improved (B of FIG. 10), but it was confirmed that only P-siSTAT3 of the present disclosure improved the invasion and activation of immune cells.

### <3-7> Confirmation of Expression Inhibition of Inflammatory Proteins by P-siSTAT3

To confirm whether P-siSTAT3 reduced the expression of inflammatory proteins, the expression of inflammatory proteins was confirmed by Western blot analysis, and the results were shown in A to L of FIG. 11. As a result, as shown in A of FIG. 11, it was confirmed that the expression of STAT3 and pSTAT3 was significantly reduced in the EAE+P-siSTAT3 group compared to the EAE group, and the expression of neuroglial markers GFAP and Iba-1 was also significantly reduced. In addition, it was confirmed that the expression of iNOS, an inflammation-mediating enzyme, was significantly reduced, and thus it was confirmed that P-siSTAT3 significantly reduced spinal cord inflammation induced by EAE. In the P-Sc group and the siRNA group, there was no significant difference in the expression of inflammatory factors from the EAE group (B of FIG. 11), and thus it was confirmed that only the P-siSTAT3 of the present disclosure inhibited the expression of inflammatory factors.

### <3-8> Confirmation of Reduction in Inflammatory Cytokines by P-siSTAT3

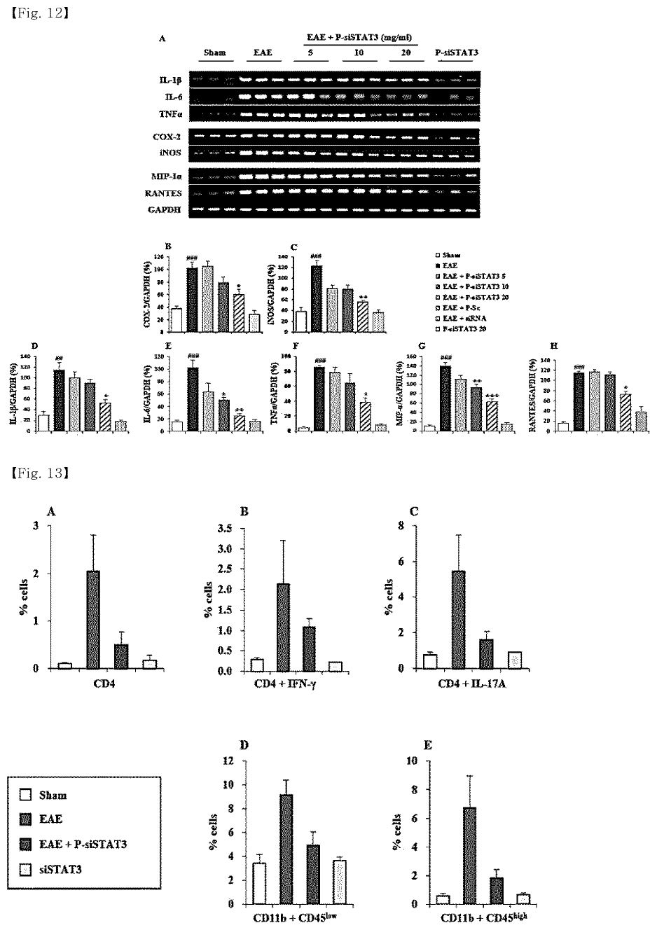

The inhibitory effects of P-siSTAT3 of the present disclosure on the mRNA expression of proinflammatory cytokines, inflammation-mediating enzymes, and monocyte-related chemokines were confirmed by RT-PCR. As a result, it was confirmed that the EAE+P-siSTAT3 group showed a significant decrease in the mRNA expression of proinflammatory cytokines (IL-1β, IL-6, and TNF-α), inflammation-mediating enzymes (COX-2 and iNOS), and leukocyte-related chemokines (MIP-1a and RANTES) compared to the EAE group, and thus it was confirmed that P-siSTAT3 significantly inhibited the inflammatory response in the spinal cord (A to H of FIG. 12).

### <3-9> Confirmation of Inhibition of T Cell Differentiation and Invasion by P-siSTAT3

To confirm the T cell differentiation and invasion inhibitory effects of P-siSTAT3 of the present disclosure, flow cytometry was performed using CD4, a T cell marker. In addition, a marker IFN-γ of Th1 cells and a marker IL-17 of Th17 were used to confirm the activity of T cells, and a marker D11b+/CD45 of both cells was used to confirm the activity and invasion of microglia and peripheral-derived macrophages. As a result, as shown in A to E of FIG. 13, the EAE+P-siSTAT3 administered group showed a decrease in the degree of invasion and activity of T cells compared to the EAE group, and the activity and invasion of microglia (CD11b⁺/CD45^{+(low)}) and macrophages (CD11b⁺/CD45^{+(high)}) were also significantly reduced.

Therefore, according to the present disclosure, it was confirmed that in an EAE animal model, which is a multiple sclerosis or encephalomyelitis animal model, the P-siSTAT3 reduced a clinical symptom index induced by EAE, and inhibited demyelination of the spinal cord. In addition, the present disclosure has been confirmed to block a STAT3 signaling pathway associated with EAE and thus reduce demyelination, and inhibit microglial and neuroglial expression. In addition, the present disclosure has been confirmed to inhibit the inflammatory factors iNOS, IL-1β, IL-6, TNF-α, and inhibit the expression of the inflammation-mediating enzyme COX-2 and the chemokines MIP-1α, RANTES. In addition, it has been confirmed to inhibit the invasion and activity of Th1 and Th17 cells associated with autoimmunity and thus to reduce autoimmune neurological disorder.

1. A method for preventing or treating autoimmune neurological disorder, comprising administering to a subject in need thereof nanoparticles consisting of STAT3-specific siRNA and a biodegradable polymer material.

2. The method of claim 1, wherein the STAT3-specific siRNA includes at least one base sequence selected from the group consisting of SEQ ID NO: 1 and SEQ ID NO: 2.

3. The method of claim 1, wherein the biodegradable polymer material is at least one selected from the group consisting of poly(lactic-co-glycolic acid) (PLGA), poly-L-lactic acid, poly-glycol acid, poly-D-lactic acid-co-glycol acid, poly-L-lactic acid-co-glycol acid, poly-D,L-lactic acid-co-glycol acid, polycaprolactone, poly-valerolacton, poly-hydroxy butyrate, poly-hydroxy valerate, dextran, poly-phosphazen, and poly-amino ester.

4. The method of claim 1, wherein the autoimmune neurological disorder is at least one selected from the group consisting of multiple sclerosis, neuromyelitis optica, acute disseminated encephalomyelitis, ascending myelitis, central myelitis, descending myelitis, transverse myelitis, myasthenia gravis, guillain-barre syndrome, autoimmune uveitis, autoimmune encephalopathy and chronic inflammatory demyelinating polyneuropathy.

5. The method of claim 1, wherein in the nanoparticle, the STAT3-specific siRNA is bound to the surface of the biodegradable polymer material.

6. The method of claim 1, wherein the nanoparticles consisting of the STAT3-specific siRNA and the biodegradable polymer material inhibit demyelination.

7. The method of claim 1, wherein the nanoparticles consisting of the STAT3-specific siRNA and the biodegradable polymer material inhibit phosphorylation of STAT3.

8. The pharmaceutical composition method of claim 1, wherein the nanoparticles consisting of the STAT3-specific siRNA and the biodegradable polymer material reduce the expression of Iba-1 or GFAP.

9. The method of claim 1, wherein the nanoparticles consisting of the STAT3-specific siRNA and the biodegradable polymer material inhibit the expression of inflammatory cytokines, inflammation-mediating enzymes, or chemokines.

10. The method of claim 9, wherein the inflammatory cytokine is at least one selected from the group consisting of IL-1β, IL-6, and TNF-α.

11. The method of claim 9, wherein the inflammation-mediating enzyme is at least one selected from the group consisting of COX-2 and iNOS.

12. The method of claim 9, wherein the chemokine is at least one selected from the group consisting of MIP-1α and RANTES.

13. The method of claim 1, wherein the nanoparticles consisting of the STAT3-specific siRNA and the biodegradable polymer material reduce the invasion or activity of immune cells.

14. The method of claim 13, wherein the immune cell is at least one selected from the group consisting of Th1 and Th17.

15. The method of claim 1, wherein the nanoparticles consisting of the STAT3-specific siRNA and the biodegradable polymer material prevent damage to a blood-brain barrier (BBB).

16-19. (canceled)


SEQUENCE LISTING (18 sequences)

SEQ ID NO: 1 - RNA, 25 nt, synthetic construct
  source 1..25: mol_type = other RNA; note = STAT3 specific siRNA 1
aaacgtgagcgactcaaactgccct

SEQ ID NO: 2 - RNA, 25 nt, synthetic construct
  source 1..25: mol_type = other RNA; note = STAT3 specific siRNA2
agggcagtttgagtcgctcacgttt

SEQ ID NO: 3 - DNA, 20 bp, synthetic construct
  source 1..20: mol_type = other DNA; note = COX-2 Forward primer
gctcggcttccagtattgag

SEQ ID NO: 4 - DNA, 20 bp, synthetic construct
  source 1..20: mol_type = other DNA; note = COX-2 Reverse primer
agaaggaaatggctgcagaa

SEQ ID NO: 5 - DNA, 21 bp, synthetic construct
  source 1..21: mol_type = other DNA; note = iNOS Forward primer
ggcaaacccaaggtctaggtt

SEQ ID NO: 6 - DNA, 20 bp, synthetic construct
  source 1..20: mol_type = other DNA; note = iNOS Reverse primer
tcgctcaagttcagcttggt

SEQ ID NO: 7 - DNA, 21 bp, synthetic construct
  source 1..21: mol_type = other DNA; note = IL-1Beta Forward primer
ttgttgctgtggagaagctgt

SEQ ID NO: 8 - DNA, 21 bp, synthetic construct
  source 1..21: mol_type = other DNA; note = IL-1Beta Reverse primer
aacgtcacacaccagcaggtt

SEQ ID NO: 9 - DNA, 21 bp, synthetic construct
  source 1..21: mol_type = other DNA; note = IL-6 Forward primer
tccatccagttgccttcttgg

SEQ ID NO: 10 - DNA, 21 bp, synthetic construct
  source 1..21: mol_type = other DNA; note = IL-6 Reverse primer
ccacgatttcccagagaacat

SEQ ID NO: 11 - DNA, 20 bp, synthetic construct
  source 1..20: mol_type = other DNA; note = TNF-alpha Forward primer
agcccccagtctgtatcctt

SEQ ID NO: 12 - DNA, 20 bp, synthetic construct
  source 1..20: mol_type = other DNA; note = TNF-alpha Reverse primer
ctccctttgcagaactcagg

SEQ ID NO: 13 - DNA, 20 bp, synthetic construct
  source 1..20: mol_type = other DNA; note = MIP-1alpha Forward primer
cagccaggtgtcattttcct

SEQ ID NO: 14 - DNA, 20 bp, synthetic construct
  source 1..20: mol_type = other DNA; note = MIP-1alpha Reverse primer
aggcattcagttccaggtca

SEQ ID NO: 15 - DNA, 20 bp, synthetic construct
  source 1..20: mol_type = other DNA; note = RANTES Forward primer
acaccactccctgctgcttt

SEQ ID NO: 16 - DNA, 22 bp, synthetic construct
  source 1..22: mol_type = other DNA; note = RANTES Reverse primer
gactgcaagattggagcacttg

SEQ ID NO: 17 - DNA, 21 bp, synthetic construct
  source 1..21: mol_type = other DNA; note = GAPDH Forward primer
aggtcatcccagagctgaacg

SEQ ID NO: 18 - DNA, 22 bp, synthetic construct
  source 1..22: mol_type = other DNA; note = GAPDH Reverse primer
caccctgttgctgtagccgtat